(19) United States
(12) Patent Application Publication
(10) Pub. No.: US 2026/0224180 A1
(43) Pub. Date: Aug. 6, 2026

(54) COMPRESSION UNIT FOR CHECKING BREAST COMPRESSION DURING MAMMOGRAPHY IMAGING

(71) Applicant: Siemens Healthineers AG, Forchheim (DE)

(72) Inventors: Marcus RADICKE, Veitsbronn (DE); Marcel BEISTER, Erlangen (DE); Christopher SYBEN, Cadolzburg (DE)

(73) Assignee: Siemens Healthineers AG, Forchheim (DE)

(21) Appl. No.: 19/530,449

(22) Filed: Feb. 5, 2026

(30) Foreign Application Priority Data
Feb. 6, 2025 (EP) 25156167.6

(51) Int. Cl. A61B 6/50; A61B 6/00; A61B 6/04

(52) U.S. Cl. CPC A61B 6/502; A61B 6/0414; A61B 6/44

(57) ABSTRACT

A compression unit for checking a compression of a breast during acquisition of a mammography image comprises a first compression plate, a second compression plate and at least one optical sensor. During the compression the breast is compressed between the first and the second compression plate. The first compression plate is embodied, during the compression, to form a first contact surface with the breast. The first compression plate comprises at least one first mirror. The at least one first mirror and the at least one optical sensor are arranged in such a way that a mirroring of at least one part of the first contact surface is able to be detected by the optical sensor.

### CROSS-REFERENCE TO RELATED APPLICATION(S)

The present application claims priority under 35 U.S.C. §119 to European Patent Application No. 25156167. 6, filed Feb. 6, 2025, the entire contents of which is incorporated herein by reference.

### Field

The present disclosure relates to a compression unit for mammography systems and in particular to a compression unit, to a mammography system and to a method for checking the compression of the breast during mammography imaging by using mirrors and optical sensors.

One or more example embodiments relates to the field of medical imaging, in particular mammography, for early detection of breast cancer. In mammography a breast is typically compressed between two compression plates and imaged with x-rays in order to create high resolution images of the breast tissue. A correct positioning and even compression of the breast are decisive for the image quality and value of the diagnostic information in the images.

### Related Art

Conventional mammography systems offer only limited possibilities for monitoring and optimizing the compression of the breast during the examination. Often users rely on visual inspection and their experience in order to assess the positioning and compression. This can lead to variations in the image quality and potentially to incorrect diagnoses.

Moreover, the restricted view, especially of a contact surface between the breast and at least one of the compression plates, renders a precise assessment of fold formation or uneven compression more difficult.

Previous approaches to improving the checking of compression, such as employing additional optical sensors, are often associated with increased complexity and costs. The integration of a number of sensors can moreover make it more difficult to manage the system and can adversely affect workflow.

### SUMMARY

An objective problem to be solved consists of providing an apparatus that makes possible improved checking and optimization of the breast compression during mammography imaging without significantly increasing the complexity of the system or adversely affecting the examination workflow in doing so.

This objective problem is solved by a compression unit, a mammography system and a method according to example embodiments.

### BRIEF DESCRIPTION OF THE DRAWINGS

Exemplary embodiments of the invention are shown in the drawings and will be described in greater detail below.

The same reference characters are used below in different figures for the same features. In the figures:

FIG. 1 shows a schematic side view of a compression unit for a mammography system according to one or more example embodiments;

FIG. 2 shows a schematic overhead view of a compression unit for a mammography system according to one or more example embodiments;

FIG. 3 shows a schematic side view of a compression unit for a mammography system according to one or more example embodiments;

FIG. 4 shows a schematic perspective view of a mammography system according to one or more example embodiments; and

FIG. 5 shows a flow diagram of a method for checking a compression of a breast during acquisition of a mammography image according to one or more example embodiments.

### DETAILED DESCRIPTION

Independent of the grammatical term usage, individuals with male, female or other gender identities are included within the term.

Features, advantages, or alternative forms of embodiment herein can be assigned to the other claimed subject matter in each case and vice versa. In other words claims for the systems can be improved with claims that are described or claimed in conjunction with the methods. In this case the functional features or the method are embodied by objective units of the system.

One or more example embodiments relates to a compression unit for checking a compression of a breast during acquisition of a mammography image. The compression unit comprises a first compression plate, a second compression plate and at least one optical sensor. During the compression the breast is compressed between the first and the second compression plate. In this situation the first compression plate is embodied to form a first contact surface with the breast during compression. In this case the first compression plate comprises at least one first mirror. The at least one first mirror and the at least one optical sensor are arranged in this case in such a way that a mirroring of at least one part of the first contact surface is able to be detected by the optical sensor.

The compression unit can serve to make possible a checked and monitored compression of the breast during a mammography examination.

A mammography image can be an x-ray image of the breast, which is used for early detection of breast cancer or of other breast diseases.

The first compression plate and the second compression plate can be flat, stable plates, which are embodied to clamp the breast between them and to compress it. The first and the second compression plate can be manufactured from a material that lets x-rays pass through it, for example from plastic or from carbon fiber, in order not to adversely affect the x-ray radiation during the imaging.

The at least one optical sensor can be an imaging device that is capable of recording optical images. The optical sensor can for example be a digital camera, a video camera, a time of flight camera, a LIDAR sensor or another light-sensitive sensor. The optical sensor can serve to record images of the compressed breast in order to monitor the quality of the compression.

The first contact surface can be the area of the breast that comes into direct contact with the first compression plate when said breast is compressed. This first contact surface can provide important information about the quality of the compression, such as for example the evenness of the pressure or the presence of folds in the skin.

The at least one first mirror can be a reflecting element, which is integrated into the first compression plate or is attached to it. The first mirror can be made of a material that both reflects optical light and/or lets x-rays pass through it, in order not to adversely affect mammography imaging. Possible materials for the first mirror can be thin metal coatings on an x-ray-transparent substrate or special dielectric mirrors.

The arrangement of the first mirror and of the optical sensor makes it possible for a mirroring of at least one part of the first contact surface to be able to be detected by the optical sensor. This means that the optical sensor can indirectly record images of the first contact surface between the breast and the first compression plate, in that it detects the reflection of this first contact surface in the at least one mirror. This arrangement can make it possible to observe areas of the breast that would otherwise not be directly visible for the optical sensor.

This configuration enables the compression unit to make an improved monitoring and checking of the breast compression possible during the mammography imaging. The capability of observing the first contact surface between the breast and the first compression plate can contribute to detecting and correcting problems such as uneven compression or skin folds, which can lead to an improved image quality and a more accurate diagnosis.

In a further advantageous form of embodiment of the compression unit the second compression plate can be embodied, during the compression, to form a second contact surface with the breast. The second compression plate can comprise at least one second mirror. The at least one second mirror and the at least one optical sensor can be arranged in such a way that a mirroring of at least one part of the second contact surface is able to be detected by the optical sensor.

This configuration makes possible a comprehensive monitoring of the compression process, in that both the first and also the second contact surface between the breast and the first and the second compression plate can be observed.

The second mirror can be integrated in a similar way to the first mirror into the second compression plate or can be arranged on said plate. The arrangement of the second mirror can be chosen so that the optical sensor obtains an optimum view of the mirrored second contact surface.

The second mirror can be embodied similarly to the first mirror.

The possibility of monitoring the first and the second contact surface at the same time can offer many advantages. A more complete assessment of the breast positioning and compression can be made possible. Potential problems such as uneven compression or fold formation can be recognized on both sides of the compressed breast. This can lead to an improved quality of the mammography imaging and to enhanced patient comfort.

The design of first and/or second compression plate can be modified in order to integrate the mirror surfaces.

This can be achieved by the use of specially coated areas on the surfaces of the first and/or second compression plate. As an alternative separate mirror elements can be built into the first and/or second compression plates. The integration of the mirror can be designed so that it does not adversely affect the primary function of the first and/or second compression plate.

If the first and/or the second mirror are embodied by Separate mirror elements, these can cover the entire surface of the first or second compression plate. As an alternative the mirror elements can be smaller than the overall surface of the first or second compression plate. In particular the first and/or the second mirror can then comprise a number of mirror elements. This number of mirror elements can then especially be arranged offset or at an angle or in a fan shape in relation to one another.

In a few forms of embodiment one of the compression plates, typically the second compression plate, can serve as a cover of an x-ray detector. In this case the second mirror can be integrated into the detector cover. This can make possible an efficient use of the available space in the mammography system and at the same time offer the additional functionality of compression monitoring.

The use of two mirrors, on the first and second compression plate in each case, can make possible a symmetrical monitoring of the compression process. This can be especially advantageous for ensuring that the breast is evenly compressed and that no areas are overlooked.

In a further advantageous form of embodiment of the compression unit the at least one optical sensor can be arranged on the first or the second compression plate.

This arrangement of the optical sensor makes possible a compact and efficient integration into the compression unit.

The placing of the optical sensor directly on the first or the second compression plate enables an optimum line of sight to the first and/or second mirror and thus to the first and/or second contact surface between the breast and the first and/or second compression plate to be guaranteed. This can lead to an improved image acquisition and quality of the check recordings.

The arrangement of the optical sensor on the first or the second compression plate can offer various advantages. On the one hand the space required for additional sensor holders can be reduced by this, which can contribute to a more compact overall design of the compression unit. On the other hand this arrangement can simplify the cabling and signal transmission, since the at least one optical sensor is positioned closer to the other electronic components of the compression unit.

Depending on the specific form of embodiment, the optical sensor can be arranged either on the first or the second compression plate. The choice of placement can depend on various factors, such as for example the geometry of the compression unit, the desired image perspective or accessibility for maintenance and calibration.

In a few forms of embodiment the optical sensor can be integrated into the surface of the first or second compression plate in order to guarantee a surface that is as smooth and interruption-free as a possible. In other forms of embodiment the optical sensor can be attached to the edge of the first or second compression plate in order to make possible a simpler installation or exchangeability.

The arrangement of the optical sensor on one of the compression plates can also offer the possibility of attaching a number of sensors at various positions of the first and/or second compression plate. This can make possible a more comprehensive coverage of the examination area and simplify the acquisition of check recordings from various angles of view.

In a further advantageous form of embodiment of the compression unit the first or the second compression plate can be a cover of an x-ray detector.

The combination of the functions of the first or second compression plate and a detector cover in a single component enables the overall construction of the mammography system to be simplified. This can lead to a more compact design, which reduces the space required by the system and can possibly facilitate handling for the medical personnel.

The integration can also offer potential advantages for image quality. Since the first or the second compression plate directly serves as a cover of the x-ray detector, the spacing between the compressed breast and the x-ray detector can be minimized. A smaller spacing can lead to an improved image sharpness, since scatter effects can be reduced.

In addition this configuration can possibly improve patient comfort. The reduction in the number of components that come into contact with the breast possibly enables the method to appear less invasive for the patient.

The first or second compression plate functioning as the detector cover can be manufactured from a material that is suitable both for the compression of the breast and also has an optimum ability to let x-ray radiation pass through it.

Possible materials can comprise special plastics or composite materials, which offer a sufficient mechanical stability for compression and at the same time cause a minimal attenuation of the x-ray radiation.

In this configuration the first or the second compression plate, which serves as a detector cover, can also be equipped with the mirror described in the earlier aspects. This makes possible the maintenance of the functionality for optical monitoring of the breast compression, while at the same time the advantages of the integrated detector cover are utilized.

In a further advantageous form of embodiment of the compression unit the at least one optical sensor can be an optical two-dimensional camera. An optical two-dimensional camera can be embodied to record two-dimensional images of the mirrored contact surfaces between the breast and the compression plates. These images can be used in order to assess the positioning and compression of the breast.

As an alternative, the at least one optical sensor can be an optical three-dimensional camera. An optical three-dimensional camera, in addition to acquiring two-dimensional images, can also acquire depth information of the mirrored contact surfaces. This can make possible a more accurate assessment of the breast form and compression.

In an alternative form of embodiment the at least one optical sensor can be a time-of-flight sensor. A time-of-flight sensor can measure the time of flight of light signals sent out in order to acquire precise distance information.

This can be especially useful in order to recognize fine differences in the surface of the breast during the compression.

As an alternative, the at least one optical sensor can be embodied as a time-of-flight sensor array. Such an array can consist of a number of time-of-flight sensors, which work together in order to cover a larger field of view and to deliver more detailed three-dimensional information about the surface of the breast.

In an alternative form of embodiment the at least one optical sensor can be a LIDAR sensor. LIDAR (Light Detection and Ranging) sensors can create high-resolution three-dimensional point clouds of the mirrored contact surfaces.

This more detailed data can be used during the mammography for a precise analysis of the breast form and compression.

The at least one optical sensor can also be embodied as a matrix sensor. A matrix sensor can consist of a plurality of individual sensor elements, which are arranged in a grid.

This can make possible an acquisition of the mirrored contact surfaces that covers a large-surface and is simultaneously more detailed.

All these sensor types can have a large field of view, which makes it possible to acquire an area of the mirrored contact surfaces between the breast and the compression plates that is as large as possible. This can contribute to reducing the number of sensors needed and at the same time guarantee a comprehensive monitoring of the breast compression.

In a further advantageous form of embodiment of the compression unit the compression unit can comprise more than one optical sensor. The number of optical sensors can be arranged in such a way as to acquire via the mirroring an area of the first and/or second contact surface that is as large as possible.

The use of a number of optical sensors can offer various advantages. A strategic arrangement of the sensors enables a more comprehensive coverage of the contact surfaces between the breast and the compression plates to be achieved. This can make it possible for a larger area of the breast surface to be monitored during the compression.

The optical sensors can for example be attached at various positions along the edges of the first and/or second compression plate. Such an arrangement can make it possible to detect the first and/or second contact surface from different angles and thus to obtain a more complete view of the compression of the breast.

The acquisition of an area of the first and/or second contact surface that is as large as possible via a number of sensors enables a more detailed analysis of the breast compression to be made possible. This can lead to improved checking of the compression process. For example irregularities in the compression, such as fold formation or uneven pressure, can possibly be detected earlier and more accurately.

The arrangement of the number of optical sensors can vary, depending on the specific design of the compression unit. The sensors can be placed symmetrically or asymmetrically in order to obtain an optimum coverage. The exact number and position of the sensors can be defined based on factors such as the size of the first and/or second compression plate, the expected breast sizes and the desired resolution of the monitoring.

The use of a number of sensors can also increase the reliability of the system. Should a sensor fail or be adversely affected by contamination, the other sensors can continue to deliver data and in this way guarantee a continuous monitoring.

The optical sensors can comprise a combination of various sensor types mentioned above. The combination of sensor types in the compression unit can make possible an improved checking and optimization of the compression process during the mammography. This can lead to a more accurate positioning of the breast, a more even compression and ultimately to an improved image quality of the mammography images.

The comprehensive acquisition of the first and/or second contact surfaces via a number of optical sensors possibly enables a more precise and more individual adaptation of the compression. This can lead to an improvement of the image quality while at the same time minimizing the discomfort for the patient.

In a further advantageous form of embodiment of the compression unit the at least one first mirror and/or the at least one second mirror can be made of an optically transparent material.

A suitable material for the optically transparent mirror can for example be a special glass or a transparent plastic that has a high optical reflectivity.

Advantageously the first and/or the second mirror simultaneously have a low x-ray absorption.

Possible materials comprise inter alia thin layers made of silicon dioxide, aluminum oxide or specific polymers with special coatings.

The optical transparency of the first and/or second mirror can be advantageous since it makes possible a visual inspection of the breast during the compression and imaging through the first and/or second compression plate. At the same time the first and/or the second mirror can fulfil their function, in that they make the contact surfaces between the breast and the first and/or second compression plate visible for the optical sensor.

The use of optically transparent materials for the first and/or second mirror enables a compression unit to be realized that makes possible both a precise optical checking of the breast compression and also a high-quality x-ray imaging. This can contribute to an improved accuracy and reliability of the mammography examination.

In a further advantageous form of embodiment of the compression unit the at least one first and/or the at least one second mirror can exhibit no x-ray absorption or only a small amount of x-ray absorption.

This property is especially advantageous when the mirrors are positioned in the beam path of the x-ray radiation. A small x-ray absorption of the mirrors can guarantee that the mirrors do not influence the mammography imaging or only have a minimal influence on it.

The small x-ray absorption can be achieved by the use of suitable materials for the mirror. For example thin x-ray-transparent substrates with a reflecting coating can be used.

The reflecting coating can be applied so thinly that it only minimally absorbs the x-ray radiation.

A further possibility consists of the use of dielectric mirrors, which consist of a number of layers of transparent materials with alternating heights and low refractive indices. This type of mirrors can be conceived so that they are highly reflective for visible light, while they remain almost transparent for x-ray radiation.

The homogeneity of the x-ray absorption of the mirrors can likewise be of importance. A homogeneous absorption over the entire surface of the mirror can contribute to minimizing artifacts or distortions in the mammography image. This can be achieved by an even thickness and composition of the mirror material.

The small x-ray absorption of the mirror materials can contribute to the image quality of the mammography image not being adversely affected. This can be especially important when the first and/or second mirror are positioned in the beam path of the x-ray source. In particular it can be ensured in this way that a higher dose of x-rays than necessary is not applied to the patient.

In an optional form of embodiment the at least one first and/or the at least one second mirror can be made of an optically transparent material and only have a low x-ray absorption.

Through the combination of the properties of optical transparency and low x-ray absorption the at least one first and/or the at least one second mirror can possibly fulfill a number of functions in the compression unit. These mirrors can be manufactured from materials that are permeable both for visible light and also for x-ray radiation, while at the same time they have a sufficient reflectivity for optical imaging. Possible materials for such mirrors can comprise thin layers of beryllium oxide, boron carbide or specific carbon nanotube composite materials. These materials can in some cases combine a high mechanical stability, low density and excellent optical properties. In addition multilayer structures consisting of alternating layers of silicon nitride and silicon dioxide can be used, which function as Bragg mirrors and at the same time have a low x-ray absorption. In some forms of embodiment diamond-like carbon coatings on an x-ray-transparent substrate can also be employed, which combines a high hardness and chemical durability with the desired optical and physical x-ray properties.

The use of mirrors with low and homogeneous x-ray absorption enables an optimum balance to be achieved between the optical monitoring of the compression process and the quality of the mammography image. This can contribute to an improved accuracy and reliability of the mammography method.

One or more example embodiments relates to a mammography system. The mammography system comprises an x-ray source, an x-ray detector and a compression unit as claimed in one of the preceding aspects.

The compression unit can be positioned between the x-ray source and the x-ray detector in such a way that a breast compressed with the compression unit is projected onto the x-ray detector by x-ray radiation emitted by the x-ray source.

The x-ray source can be an apparatus which creates x-ray radiation. The x-ray source can for example comprise an x-ray tube. The x-ray tube can generate a stream of electrons, which strikes an anode and, in doing so, emits x-ray radiation. The x-ray source can make various settings possible for the radiation intensity and energy, in order to guarantee optimum imaging conditions for different breast densities and thicknesses.

The x-ray detector can be an apparatus that detects the x-ray radiation transmitted through the breast and converts it into a digital image. The x-ray detector can for example be a flat panel detector, which consists of a matrix of detector elements. Each detector element can convert the incident x-ray radiation into an electrical signal, which is then digitized. The x-ray detector can have a high spatial resolution and a large dynamic range, in order to image fine details in the breast structure.

The compression unit can, as described in the preceding aspects, comprise a first and a second compression plate, between which the breast is compressed. The compression unit can additionally comprise at least one optical sensor and at least one first and/or at least one second mirror that make it possible to check the breast compression.

The arrangement of the x-ray source, the compression unit and the x-ray detectors can be designed so that an optimum imaging of the compressed breast is made possible. The x-ray source can be positioned above the compression unit, in order to send the x-ray radiation from above through the compressed breast. The x-ray detector can be arranged below the compression unit, in order to detect radiation transmitted through the breast.

In an optional form of embodiment the mammography system can be arranged to carry out tomosynthesis imaging. In tomosynthesis the x-ray source can be moved in a restricted angular range around the compressed breast, while a number of projection images are recorded from various angles. These projection images can subsequently be reconstructed into a three-dimensional image volume.

In this case the compression unit can be designed so that it holds the breast stably in position during the entire imaging sequence. The optical sensors and mirrors of the compression unit can in this case possibly be used to monitor the stability of the compression during the movement of the x-ray source. This can contribute to minimizing movement artifacts in the reconstructed tomosynthesis images.

The x-ray detector can be optimized for tomosynthesis imaging, in order to make a fast readout speed and a high dynamic possible. In a few forms of embodiment a large-area flat panel detector can be used, which covers the entire image field and in this way makes possible the acquisition of all projection images without mechanical movement of the detector.

This configuration of the mammography system can offer a number of advantages. On the one hand the integration of the compression unit with optical sensors and mirrors enables a precise checking of the breast compression to be made possible during the imaging. This can lead to an improved image quality and a reduced radiation load for the patient. On the other hand the arrangement can make an efficient execution of the mammography possible, since the compression and the imaging are integrated into a single system.

In a further advantageous form of embodiment of the mammography system the first or the second compression plate can form a cover of an x-ray detector.

The cover of the x-ray detector can be made of a material that lets x-rays pass through it, in order to guarantee the lowest possible adverse effect on the image quality. At the same time this cover can be robust enough to function as a first or second compression plate and exert the pressure required for the compression on the breast.

The choice of material for the integrated compression plate/detector cover can take account of various aspects. For example composite materials can be used, which have both a sufficient mechanical stability for the compression and also a high x-ray transparency.

The combination of the functions of first or second compression plate and detector cover into a single component enables the overall construction of the mammography system to be simplified. This can lead to a more compact design, which reduces the space required by the system and can possibly make handling easier for the medical personnel.

The integrated solution can also contribute to minimizing the distance between the compressed breast and the x-ray detector. A smaller distance can potentially improve the image sharpness, since scatter effects can be reduced.

In addition this configuration can offer the possibility of integrating sensors or other components directly into the compression plate/detector cover. For example optical sensors or mirrors for the monitoring of the breast compression can be embedded into this component without taking up any additional space.

The integration can also be advantageous from a hygiene point of view, since there are fewer separate surfaces to be cleaned and disinfected. This can simplify the maintenance and upkeep of the system.

For the patient this integrated solution can possibly lead to a more comfortable examination experience, since the number of components that come into contact with the breast are reduced.

One or more example embodiments relates to a method for checking a compression of a breast during acquisition of a mammography image with a mammography system. The method comprises the following steps:
positioning the breast between the first and the second compression plate,
compressing the breast between the first and the second compression plate,
acquiring a check recording with the at least one optical sensor,
providing the check recording,
checking the compression of the breast based on the check recording.

The positioning of the breast between the first and the second compression plate can be undertaken manually by specialist medical personnel or be automated by a positioning unit of the mammography system. The breast can be aligned in this case so that an optimal imaging of the breast tissue is guaranteed in the later mammography imaging.

The compressing of the breast between the first and the second compression plate can be carried out by a compression unit of the mammography system. The compression can be undertaken step-by-step, in order to achieve an even distribution of the breast tissue and avoid the formation of folds.

The acquisition of a check recording with the at least one optical sensor can be carried out during or immediately after the compression. The check recording can depict a mirroring of at least one part of the first contact surface on the at least one first mirror and/or a mirroring of at least one part of the second contact surface on the at least one second mirror. The optical sensor can for example be a camera, a time-of-flight sensor or a LIDAR sensor.

The provision of the check recording can comprise the transmission of the image to an evaluation unit of the mammography system. The check recording can be stored in a suitable image format and be prepared for further processing.

The checking of the compression of the breast based on the check recording can be undertaken by an evaluation unit of the mammography system. In this analysis the check recording can be analyzed for features such as even compression, absence of folds or optimum positioning of the breast. The evaluation can be automated by image processing algorithms or be performed manually by specialist medical personnel.

The individual method steps can be repeated in a loop until an optimum compression and positioning of the breast is achieved. With a negative result of the check there can be a correction of the positioning and/or of the compression of the breast, followed by a renewed acquisition and check. With a positive result the method can be continued with the acquisition of the actual mammography image.

The method described can make possible an improved quality of the mammography images, in that it ensures an optimum compression and positioning of the breast before the actual image imaging. This can lead to an increased diagnostic accuracy and a reduction in repeat images.

In a further advantageous form of embodiment of the method, with a negative result of the check, there can be a correction of the positioning and/or the compression of the breast. Subsequently the acquisition of the check recording, provision of the check recording and checking of the compression steps can be repeated.

The correction of the positioning can for example comprise a repositioning of the breast between the first and the second compression plate. Here the breast can be aligned so that, where possible, the entire breast tissue is detected and fold formation is avoided. The correction of the compression can include an adjustment of the compression pressure. The compression pressure can be increased in order to achieve a better tissue resolution, or be reduced in order to improve the patient's comfort. As an alternative or in addition, during correction of the compression, the breast can be compressed again, in which case it should be ensured that no folds are formed.

After the correction a new check recording is acquired. This new check recording can show the effects of the correction measures and makes a new assessment of the breast compression and positioning possible. The provision and checking of the new check recording is undertaken in a similar way to the previous steps.

This iterative process of correction and rechecking can be repeated until such time as a satisfactory result is reached. This feedback loop enables the quality of the mammography imaging to be improved and at the same time the patient's comfort to be optimized.

With a positive result of the checking there can be a provision of the positive result. The positive result can show that the breast is optimally positioned and compressed, in order to make possible a high-quality mammography imaging. The positive result can be provided for example by a visual or acoustic signal to the person operating the mammography system.

The provision of the positive result can be taken as an indicator that the preparations for the actual mammography imaging are completed and that the process can continue with the x-ray imaging. It can thus be ensured that the mammography imaging is being carried out under optimum conditions in respect of breast compression and positioning.

In a further advantageous form of embodiment of the method the method step of providing the positive result can initiate the following method step: Acquisition of the mammography imaging of the breast.

The provision of the positive result can serve as a trigger for the start of the actual mammography imaging process. This positive result can show that the breast is correctly positioned and compressed, based on the preceding checking of the compression via the check recording.

The initiation of the acquisition of a mammography imaging can comprise various steps. For example a signal can be sent to the x-ray source in order to start the emission of x-ray radiation. At the same time the x-ray detector can be activated in order to detect the radiation transmitted through the breast.

The transition from the compression check to the actual mammography imaging can occur automatically as soon as the system registers a positive result. As an alternative a manual confirmation step can be required by specialist medical personnel before the mammography imaging is initiated.

This method of operation enables it to be ensured that the mammography imaging only takes place when the optimum positioning and compression of the breast is achieved. This can lead to an improved image quality and reduce the need to repeat images. What is more, this method can contribute to minimizing the radiation load for the patient, since the likelihood of technically unsatisfactory images is reduced.

The acquisition of the mammography imaging can take various imaging parameters into account, which have been optimized based on the results of the compression check. For example the exposure time, the radiation dose or the imaging angle can be adjusted in order to obtain the best possible image quality.

One or more example embodiments relates to the use of a compression unit described above in a mammography system.

In an advantageous way the compression unit can be employed in a mammography system in order to check and to optimize the compression of the breast during the mammography examination. The integration of the compression unit with its optical monitoring facilities enables the overall performance and reliability of mammography examinations to be improved.

The compression unit can be positioned between an x-ray source and an x-ray detector of the mammography system. In this arrangement the compression unit can compress the breast, while at the same time the x-ray radiation is projected by the x-ray source through the compressed breast onto the x-ray detector.

The at least one optical sensor of the compression unit can acquire at least one check recording during and/or after the compression process. These check recordings can be mirror images of the first and/or second contact surface between the breast and the first and/or second compression plate. Based on this, at least one check recording the compression of the breast can be monitored and where necessary adjusted.

The use of the compression unit in a mammography system can offer a number of advantages. The continuous optical monitoring can guarantee an even and appropriate compression of the breast. This can lead to an improved image quality of the mammography images, since an optimum compression reduces the tissue thickness and can thus increase the contrast and the ability to recognize detail in the x-ray images.

What is more, the use of the compression unit with optical monitoring can improve patient comfort. The precise checking of the compression enables is disproportionate or uneven compression to be avoided, which can reduce the discomfort for the patient during the examination.

The integration of the compression unit into the mammography system can also optimize the workflow for the specialist medical personnel. The automatic monitoring and checking of the breast positioning and compression can reduce the need for manual adjustments and thus enhance the efficiency of the examination.

Overall, the use of the compression unit in a mammography system can contribute to more accurate diagnoses, improved patient comfort and more efficient workflows, which can enhance the overall quality and reliability of mammography examinations.

Moreover, the use of the compression unit in a mammography system can contribute to reducing a radiation dose applied, since incorrect images can be avoided.

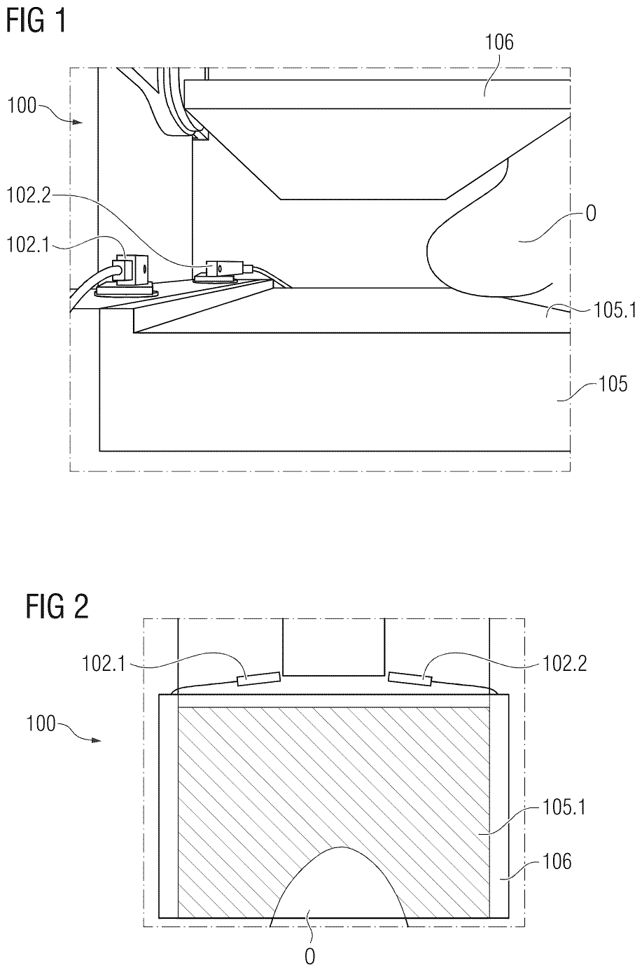

FIG. 1 shows a side view of a compression unit 100, in which the arrangement of the components for breast examination and compression can be seen. Arranged on the upper side is a first compression plate that is in particular optical 106 and/or transparent for x-ray radiation, which can be embodied to compress the breast O during the examination. A second compression plate can be arranged below the first compression plate 106. The second compression plate can be embodied as a cover 105.1 of an x-ray detector 105, which can serve as protection for the x-ray detector 105 positioned below it.

The compression unit 100 can comprise at least one optical sensor 102.1, 102.2 for monitoring the compression process. A first optical sensor 102.1 can be arranged on the left side of the compression unit 100 in order to acquire images of the breast compression area. A second optical sensor 102.2 can likewise be comprised by the compression unit 100.

The x-ray detector 105 is embodied to acquire x-ray images during a mammography imaging.

The space between the first compression plate 106 and the cover 105.1 of the x-ray detector 105 can be the area in which the breast 0 can be positioned during the imaging. The optically transparent property of the first compression plate 106 can make a visual inspection of the breast positioning and compression possible.

The overall design of the compression unit 100 can integrate compression, imaging and monitoring capabilities in order to make precise and checked mammography examinations possible.

The arrangement of the components in the compression unit 100 can make possible a simultaneous breast compression, x-ray imaging and optical monitoring of the compression process. The first compression plate 106 and/or the cover 105.1 of the x-ray detector 105 can comprise at least one first and/or one second mirror. The first and/or the second optical sensor 102.1, 102.2 can acquire at least one reflected image (check recording) of a first and/or second contact surface of the breast O with the first compression plate 106 and/or the cover 105.1 of the x-ray detector 105.

FIG. 2 shows a schematic diagram of an advantageous form of embodiment of a compression unit 100 for a mammography system in a view from above. The compression unit 100 can comprise a first compression plate 106 and a second compression plate. In the view from above the first compression plate 106 can be seen as a transparent plate, which can be embodied during the compression to form a first contact surface with a breast O.

Arranged below the first compression plate 106 can be a cover 105.1 of an x-ray detector 105. This cover 105.1 can serve as a second compression plate and can be embodied, during the compression, to form a second contact surface with the breast o.

The space between the first compression plate 106 and the cover 105.1 of the x-ray detector 105 can represent the area in which the breast O can be positioned during the imaging.

The compression unit 100 can comprise at least one optical sensor 102.1, 102.2. In an advantageous form of embodiment two optical sensors 102.1, 102.2 can be provided. A first optical sensor 102.1 can be arranged on the left side of the compression unit 100, while a second optical sensor 102.2 can be positioned on the right side. This arrangement can make possible a comprehensive monitoring of the compression area of the breast O.

The first compression plate 106 can comprise at least one first mirror. In a similar way the second compression plate can comprise at least one second mirror. The at least one first mirror and the at least one second mirror can be arranged in such a way that a mirroring of at least one part of the first contact surface and/or of the second contact surface by the at least one first and/or second optical sensor 102.1, 102.2 is able to be acquired.

The optically transparent nature of the first compression plate 106 can make a visual inspection of the breast positioning and compression possible. This can lead to an improved checking and adjustment of the compression process.

The overall design of the compression unit 100 can integrate compression, imaging and monitoring capabilities in order to make precise and checked mammography examinations possible.

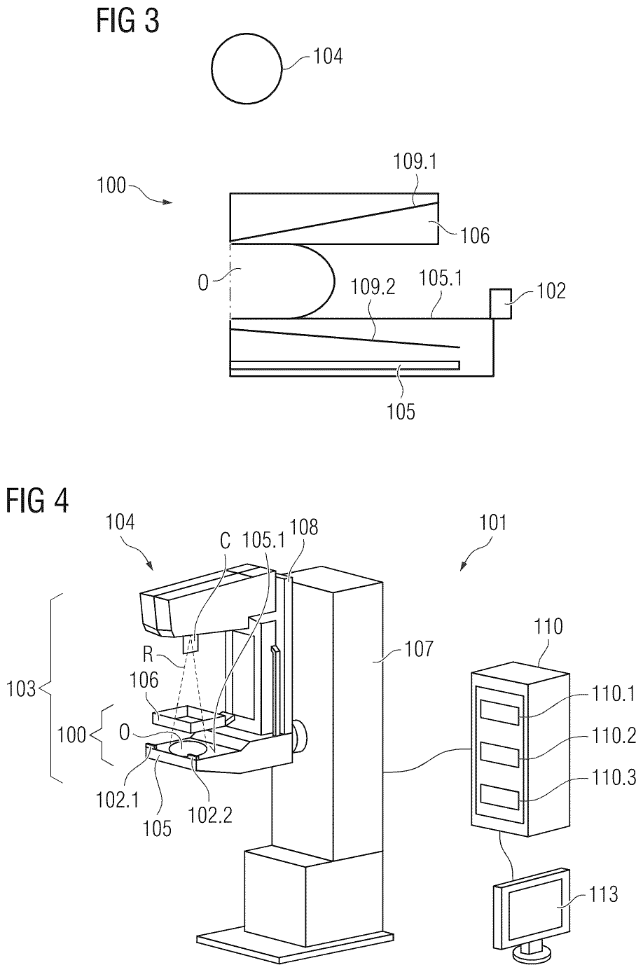

FIG. 3 shows a schematic diagram of an advantageous form of embodiment of a compression unit 100 for checking a compression of a breast O during acquisition of a mammography image.

The compression unit 100 can comprise a first compression plate 106 and a second compression plate. The first compression plate 106 can be embodied as the upper compression plate and the second compression plate can be embodied as the lower compression plate. A breast O can be positioned and compressed between the first compression plate 106 and the second compression plate.

The second compression plate can be embodied as the cover 105.1 of an x-ray detector 105. The compression plate functioning as the cover 105.1 of the x-ray detector 105 can be made from a material that is both suitable for the compression of the breast O and also has an optimum ability to let x-rays pass through it.

The first compression plate 106 can comprise a first mirror 109.1. The first mirror 109.1 can be integrated into the first compression plate 106. The second compression plate can comprise a second mirror 109.2. The second mirror 109.2 can be integrated into the second compression plate.

The compression unit 100 can furthermore comprise at least one optical sensor 102. The optical sensor 102 can be arranged on the second compression plate. The optical sensor 102 can for example be an optical camera, a time-of-flight sensor or a LIDAR sensor.

Arranged above the first compression plate 106 can be an x-ray source 104. The x-ray source 104 can be embodied to emit x-ray radiation R in the direction of the compressed breast O . Arranged below the second compression plate can be an x-ray detector 105. The x-ray detector 105 can be embodied to detect the x-ray radiation R transmitted through the compressed breast O.

The first mirror 109.1 in the first compression plate 106 and the second mirror 109.2 in the second compression plate can be arranged in such a way that mirror images of parts of the first and/or second contact surface between the compressed breast O and the first or second compression plate 106 can be detected by the optical sensor 102. The first mirror 109.1 and the second mirror 109.2 can be made of an optically transparent material and have a low x-ray absorption.

The arrangement of the mirror 109.1, 109.2 and the optical sensor 102 enables a visual check of the compression of the breast O to be made possible. The optical sensor 102 can detect images of the mirrored first and/or second contact surface. These images can be used to assess the quality of the compression and for recognizing possible problems such as the formation of folds or uneven compression.

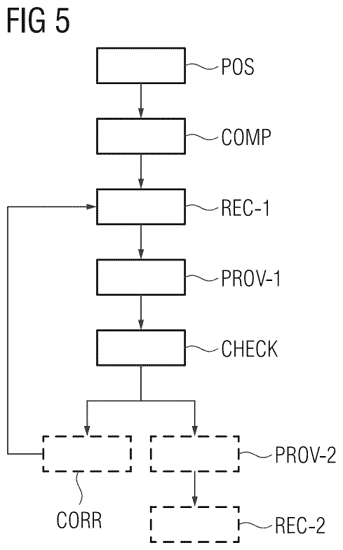

FIG. 4 shows a mammography system 101 comprising a compression unit 100 described above.

The mammography system 101 can comprise a source detector arrangement 103. The source-detector arrangement 103 can comprise an x-ray source 104 and an x-ray detector 105. The x-ray source 104 can be embodied to create x-ray radiation R and emit it in the direction of the x-ray detector 105. The x-ray detector 105 can be embodied to detect the x-ray radiation R, transmitted by the breast o to be examined and to convert it to electrical signals.

A compression unit 100 can be arranged between the x-ray source 104 and the x-ray detector 105. The compression unit 100 can be embodied as claimed in the description for FIGS. 1 to 3. The compression unit 100 can comprise a first compression plate 106 and a second compression plate. The first compression plate 106 and second compression plate can be embodied in such a way as to compress a breast O during a mammography examination. The compression unit 100 can furthermore comprise at least one optical sensor 102. The optical sensor 102 can be embodied to detect check recordings of the compressed breast O in the form of mirror images on the first and/or second mirror.

The mammography system 101 can comprise a pillar 107, to which a swivel arm 108 can be attached. The swivel arm 108 can bear the source detector arrangement 103 and the compression unit 100. The attachment to the pillar 107 enables a rotation of the source detector arrangement 103 and the compression unit 100 about a vertical axis to be made possible. This can allow the recording of mammography images from various angles.

The mammography system 101 can be designed to carry out tomosynthesis imaging. In tomosynthesis the swivel arm 108 can move the x-ray source 104 in a restricted angular range about the compressed breast O, while a number of projection images are acquired from various angles. These projection images can subsequently be reconstructed into a three-dimensional image volume.

The swivel arm 108 can be constructed so that it makes possible a precise and stable movement of the x-ray source 104 during the tomosynthesis images. The movement of the swivel arm 108 can be controlled by a drive mechanism, which guarantees an even and repeatable rotation.

During the tomosynthesis imaging the compression unit 100 can hold the breast O in a stable position. The optical sensors 102.1 and 102.2 can possibly be used in order to monitor the stability of the compression during the movement of the x-ray source 104. This can contribute to minimizing movement artifacts in the reconstructed tomosynthesis images.

The x-ray detector 105 can be optimized for the tomosynthesis imaging in order to make a fast readout speed and a high dynamic possible. In a few forms of embodiment a large-surface flat-panel detector can be used, which covers the entire image field and in this way makes possible the acquisition of all projection images without mechanical movement of the detector.

The control of the tomosynthesis images, including the movement of the swivel arm 108, the activation of the x-ray source 104 and the synchronization with the x-ray detector 105, can be coordinated by a control device. The control device 110 can also undertake the reconstruction of the tomosynthesis data into a three-dimensional image volume.

The mammography system 101 can comprise a control device 110. The control device 110 can comprise an interface 110.1, a processing unit 110.2 and a memory unit 110.3. The interface 110.1 can be used for communication with other components of the mammography system 101. The processing unit 110.2 can be embodied for processing of data and control of the mammography system 101. The memory unit 110.3 can be used for storage of data, images and control programs.

The mammography system 101 can furthermore comprise a terminal 113. The terminal 113 can provide a user interface, via which an operator can control the mammography system 101 and can display examination results and/or check recordings.

The various components of the mammography system 101 can work together in order to carry out a mammography examination with integrated compression checking. The compression unit 100 can compress the breast O, while the optical sensor 102 detects check recordings. These check recordings can be analyzed by the processing unit 110.2 of the control device 110 in order to monitor the quality of the compression. With satisfactory compression the x-ray source 104 can be activated in order to create a mammography image, which is detected by the x-ray detector 105. The detected data can be stored in the memory unit 110.3 and be displayed via the terminal 113.

FIG. 5 shows a schematic diagram of an advantageous form of embodiment of a method for checking a compression of a breast O during acquisition of a mammography image.

Method steps depicted by dashed lines are optional.

The method can comprise the following steps:

The method can begin with a positioning POS of the breast O between a first and a second compression plate 106.

The breast O can be placed in this case so that an optimum imaging is made possible. This step can be carried out manually by specialist medical personnel or automatically by a positioning unit of the mammography system 101.

After the positioning POS there can be a compression COMP of the breast O between the first and the second compression plate 106. The compression COMP can serve to reduce the thickness of the breast and spread the breast tissue in order to obtain an improved image quality. This step can be carried out by a compression unit 100 (for example in accordance with FIGS. 1 to 3) of the mammography system 101, which increases the pressure step-by-step in order to obtain an even distribution of the breast tissue.

Following on from the compression COMP and/or even during the compression COMP an acquisition REC-1 of a check recording with at least one optical sensor 102 can be carried out. The check recording can depict the mirroring of at least one part of a first contact surface on at least one first mirror 109.1 and/or a mirroring of at least one part of a second contact surface on at least one second mirror 109.2.

These mirrorings can make it possible to visualize the first and/or second contact surface between the breast O and the first and/or second compression plate 106. The optical sensor 102 can for example be a camera, a time-of-flight sensor or a LIDAR sensor, which acquires the image automatically.

After the acquisition REC-1 there can be a provision PROV-1 of the check recording. The check recording can be made available for a further analysis. This step can comprise the transmission of the check recording to an evaluation unit of the mammography system 101, which stores and prepares the data in a suitable format. As an alternative or in addition the provision PROV-1 can comprise a display of the check recording on a monitor or screen for a medical specialist and/or an operator of the mammography system 101.

A check CHECK of the compression of the breast O can be carried out based on the check recording. In this case it can be checked whether the breast O is correctly positioned and compressed. This evaluation can be undertaken automatically by image processing algorithms of the evaluation unit or manually by specialist medical personnel, who analyze the image for features such as even compression or absence of folds.

With a negative result of the check CHECK there can be a correction CORR of the positioning and/or of the compression of the breast O. This can include a repositioning of the breast O and/or an adaptation of the compression pressure and/or of the compression itself by the compression unit 100 and/or by specialist medical personnel. Subsequently the steps of acquisition REC-1 of the check recording, of provision PROV-1 of the check recording and of checking CHECK the compression are repeated. This iterative process can be continued until a satisfactory result is achieved.

With a positive result of the checking CHECK there can be a provision PROV-2 of the positive result. This can signal that the breast O is positioned and compressed optimally for the mammography image. The provision of the positive result can for example be indicated by a visual or acoustic signal to the operator of the mammography system.

The provision PROV-2 of the positive result can initiate an acquisition REC-2 of a mammography image of the breast O. The mammography image can then be carried out under optimum conditions. For this a signal can be sent to the x-ray source 104 in order to start the emission of x-ray radiation, while at the same time the x-ray detector 105 is activated in order to detect the transmitted radiation.

Through this method an improved quality and consistency of mammography images can be achieved. The possibility of correction CORR before the actual mammography image can contribute to reducing repeated imaging and to minimizing the radiation load for the patient. The method can be carried out both manually by trained medical personnel and also in an automated manner by the various components of the compression unit 100 and/or of the mammography system 101, wherein a combination of manual and automated steps is possible.

Where not already explicitly described, individual forms of embodiment or their individual aspects and features can be combined with one another or exchanged, without restricting or expanding the scope of the invention described, provided such a combination or such an exchange is sensible and in the spirit of this invention. Advantages that are described in relation to a form of embodiment of the present invention, are, where applicable, also advantageous for other forms of embodiment of the present invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or, ” includes any and all combinations of one or more of the associated listed items. £ The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions of operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc. ), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices) ; volatile memory devices (including, for example static random access memory devices or a dynamic random access memory devices) ; magnetic storage media (including, for example an analog or digital magnetic tape or a hard disk drive) ; and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A compression unit for checking a compression of a breast during acquisition of a mammography image, the compression unit comprising:
a first compression plate including at least one first mirror;
a second compression plate; and
at least one optical sensor,
wherein, during the compression,
the breast is compressed between the first and the second compression plate,
the first compression plate forms a first contact surface with the breast, and
the at least one first mirror and the at least one optical sensor are arranged such that a mirroring of at least one part of the first contact surface is detectable by the optical sensor.

2. The compression unit of claim 1, wherein
the second compression plate is configured, during the compression, to form a second contact surface with the breast,
the second compression plate comprises at least one second mirror, and the at least one second mirror and the at least one optical sensor are arranged such that a mirroring of at least one part of the second contact surface is detectable by the optical sensor.

3. The compression unit of claim 1, wherein the at least one optical sensor is on the first compression plate or the second compression plate.

4. The compression unit of claim 1, wherein the first compression plate or the second compression plate is a cover of an x-ray detector.

5. The compression unit of claim 1, wherein the at least one optical sensor is an optical two-dimensional camera, an optical three-dimensional camera, a time-of-flight sensor, a time-of-flight sensor array, or a LIDAR sensor.

6. The compression unit of claim 2, further comprising:
more than one optical sensor, wherein a number of optical sensors are arranged in such a way as to detect via the mirroring an area of at least one of the first contact surface or second contact surface.

7. The compression unit of claim 2, wherein at least one of the at least one first mirror or the at least one second mirror are made of an optically transparent material.

8. The compression unit of claim 2, wherein at least one of the at least one first mirror or the at least one second mirror exhibit no x-ray absorption or only a low x-ray absorption.

9. A mammography system comprising:
an x-ray source;
an x-ray detector; and
the compression unit of claim 1, wherein the compression unit is between the x-ray source and the x-ray detector.

10. The mammography system of claim 9, wherein the first compression plate or the second compression plate forms a cover of the x-ray detector.

11. A method for checking a compression of a breast during acquisition of a mammography image with the mammography system of claim 9, the method comprising:
positioning the breast between the first compression plate and the second compression plate;
compressing the breast between the first compression plate and the second compression plate;
acquiring a check recording with the at least one optical sensor, wherein the check recording records at least one of (i) the mirroring of at least one part of the first contact surface on the at least one first mirror or (ii) the mirroring of at least one part of the second contact surface on the at least one second mirror; and
checking of the compression of the breast based on the check recording.

12. The method of claim 11, wherein for a negative result of the check, the method further comprises,
correcting at least one of the positioning or the compression of the breast and repeating of the acquiring and the checking, and
for a positive result, the method further comprises, providing the positive result.

13. The method of claim 12, further comprising:
acquiring a mammography image of the breast after the provision of the positive result.

14. A mammography system comprising:
the compression unit of claim 1.

15. The compression unit of claim 2, wherein the at least one optical sensor is on the first compression plate or the second compression plate.

16. The compression unit of claim 15, wherein the first compression plate or the second compression plate is a cover of an x-ray detector.

17. The compression unit of claim 16, wherein the at least one optical sensor is an optical two-dimensional camera, an optical three-dimensional camera, a time-of-flight sensor, a time-of-flight sensor array, or a LIDAR sensor.

18. The compression unit of claim 17, further comprising:
more than one optical sensor, wherein a number of optical sensors are arranged in such a way as to detect via the mirroring an area of at least one of the first contact surface or second contact surface.

19. The compression unit of claim 18, wherein at least one of the at least one first mirror or the at least one second mirror are made of an optically transparent material.

20. The compression unit of claim 19, wherein at least one of the at least one first mirror or the at least one second mirror exhibit no x-ray absorption or only a low x-ray absorption.
